(19) United States
(12) Patent Application Publication
(10) Pub. No.: US 2006/0225744 A1
(43) Pub. Date: Oct. 12, 2006

(54) System and method for promoting oral health

(75) Inventors: Laura F. Braunecker, Alpharetta, GA (US); Eric F. Wagner, Sherwood, WI (US); John Gavin MacDonald, Decatur, GA (US)

Correspondence Address: SCHWEGMAN, LUNDBERG, WOESSNER & KLUTH, P.A., P.O. BOX 2938, MINNEAPOLIS, MN, 55402, US

(73) Assignee: Kimberly-Clark Worldwide, Inc.

(21) Appl. No.: 11/103,709

(22) Filed: Apr. 12, 2005

(51) Int. Cl. A61B 19/00; A61B 5/08

(52) U.S. Cl. 128/897; 600/530

(57) ABSTRACT

The present invention provides a low-cost system and method for detecting substances within a mouth that influence oral health. The system and method also treat the mouth when certain substances are detected within the mouth. In some forms, the system includes a holder, a sensor that is attached to the holder and an oral treatment that is secured by the holder. The sensor detects a substance that influences oral health when the system is placed near a mouth. The oral treatment engages the mouth to treat the mouth when the substance is detected within the mouth. The method includes placing the system near a mouth and detecting the presence of a substance that influences oral health using the sensor. The method further includes treating the mouth using the oral treatment.

### FIELD OF THE INVENTION

The present invention relates to promoting oral health, and more specifically to a system and method that detect the presence of substances within a mouth and then treat the mouth when certain substances are detected within the mouth.

### BACKGROUND OF THE INVENTION

The ability to reliably monitor the presence of substances that cause halitosis, gingivitis, plaque, tartar, cancer and viral infections (e.g., herpes simplex) is a key element in treating oral heath ailments. Historically, people have visited their dentist once or twice a year in order to learn about the effectiveness of their oral hygiene habits. Unfortunately, these periodic visits to the dentist may not happen often enough to accurately indicate the presence of harmful substances in the mouth. The desire to reduce breath odor and disease has made developing convenient, accurate, and reliable devices for providing oral healthcare a goal of healthcare professionals and researchers.

As an example, halitosis affects the quality of life of millions of people each year by contributing to embarrassing encounters and psychological stress. In addition, the bacterium that causes halitosis can lead to unsightly tartar, inflammation of the gums and even periodontal disease when such bacterium goes undetected and untreated.

A variety of different types of mouth treatments are typically used to improve breath odor and/or reduce the amount of harmful substances within a mouth (e.g., mouthwashes and/or breath mints). One drawback with many of these treatments is that they often mask the origin of a problem (i.e., the presence of damaging bacteria) without informing the user that there is actually a problem.

One known device for detecting oral bacteria or other harmful substances is a halitosis detector device (see, e.g., U.S. Pat. No. 4,823,803). Most halitosis detector devices typically include a chamber that receives exhalation to be tested from a tube. A sensor, which detects malodorant gases of predetermined chemical compositions, is typically located in the chamber to detect the concentration of the malodorant gases within the exhalation. In addition, there are some known portable devices that detect bad breath.

One drawback with existing halitosis detector devices is that they are used separately from any type of treatment activity thereby making such devices cumbersome and impractical for daily use.

### SUMMARY OF THE INVENTION

The present invention provides a low-cost system and method for detecting substances within a mouth that influence oral health. The system and method also treat the mouth when certain substances are detected within the mouth.

In some forms, the system includes a holder and a sensor that is attached to the holder. The sensor is adapted to detect a substance (e.g., bacteria) that influences oral health when the system is placed in (or near) a mouth. The system further includes an oral treatment that is secured by the holder. The oral treatment engages the mouth to treat the mouth when the substance is detected within the mouth.

In some embodiments, the oral treatment may be a breath freshener (e.g., a mint). In addition, the oral treatment may be a liquid, gum, aerosol spray, strip, disk or capsule (among other forms).

In some forms, the method includes placing a system near a mouth. The system includes a holder, a sensor that is attached to the holder and an oral treatment that is secured by the holder. The method further includes detecting the presence of a substance that influences oral health using the sensor and treating the mouth using the oral treatment.

In some embodiments, placing a system near a mouth includes placing the holder within the mouth. In addition, detecting the presence of the substance that influences oral health may include detecting gases and/or bacteria (among other substances) within the mouth.

### BRIEF DESCRIPTION OF THE DRAWINGS

The above-mentioned and other features will become better understood by reference to the following description of the invention and the accompanying drawings.

FIG. 1 illustrates an example system that may be used to detect and treat substances that influence oral health.

FIG. 2 illustrates example electronic circuitry that may be incorporated into a system of the present invention.

FIG. 3 illustrates another system that may be used to detect and treat substances which influence oral health.

FIG. 4 illustrates another system that may be used to detect and treat substances which influence oral health.

FIG. 5 illustrates another system that may be used to detect and treat substances which influence oral health.

FIG. 6 illustrates another system that may be used to detect and treat substances which influence oral health.

FIG. 7 illustrates another system that may be used to detect and treat substances which influence oral health.

FIG. 8 illustrates an example method of detecting and treating substances that influence oral health.

### DESCRIPTION OF THE INVENTION

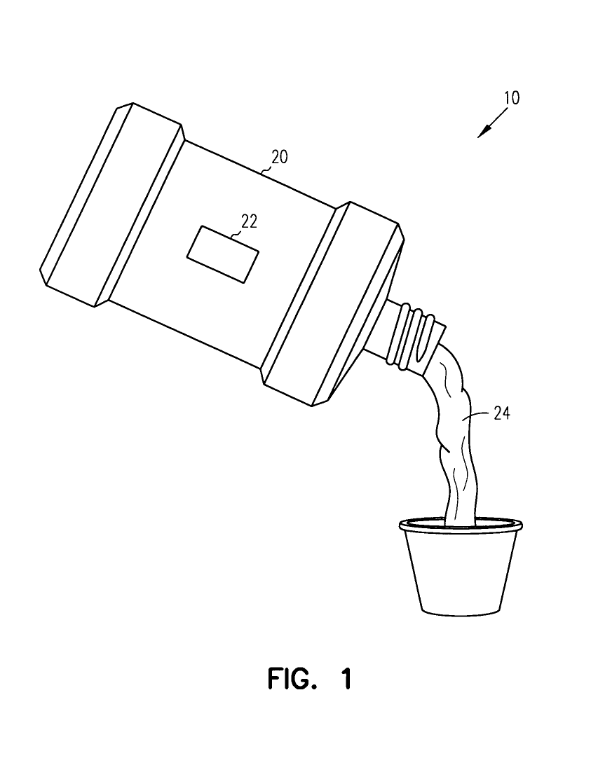

FIG. 1 illustrates an example system 10 of the present invention. The system 10 includes a holder 20 and a sensor 22 that is attached to the holder. The system further includes an oral treatment 24 that is secured by the holder 20.

The sensor 22 is adapted to detect substances that influence oral health when the system 10 is placed near a mouth. In addition, the oral treatment 24 is adapted to treat the mouth when the sensor 22 detects substances within the mouth.

In the example embodiment illustrated in FIG. 1, the holder 20 is a container (e.g., a bottle) and the oral treatment 24 is a mouthwash in liquid form. It should be noted that although holder 20 is shown as being a bottle, holder 20 may be any type of container. In addition, the mouthwash may be in liquid, solid or paste form.

Sensor 22 may be designed to collect specific molecules related to oral health. The sensor 22 may be integrated into the holder 20 such that the sensor 22 is exposed to a user's saliva and/or breath by positioning the sensor near (or within) the user's mouth. In some embodiments, the sensor 22 may be detachably connected to the holder 20.

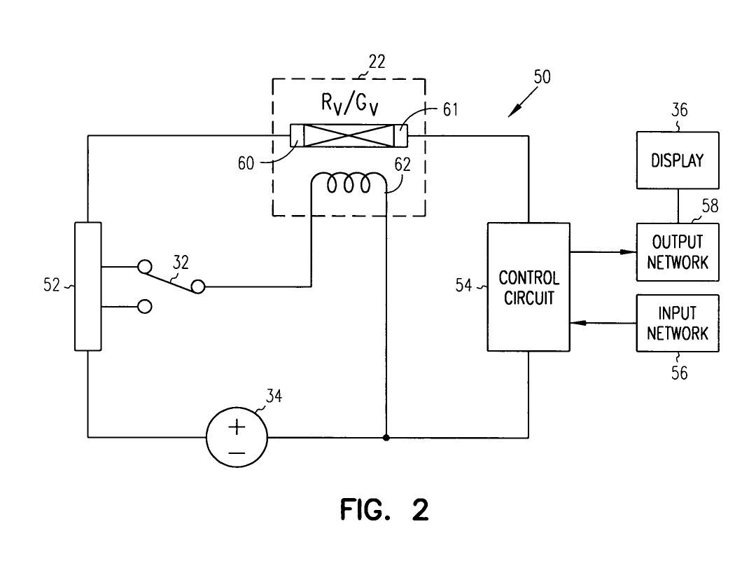

As shown in FIG. 2, the sensor 22 may work in conjunction with a switch 32, a power source 34, and a display 36. The switch 32 may be a bipolar switch with an “on” position and an “off” position. The switch 32 may be integrated into the holder 20 such that a user is able to readily operate the system 10.

Power source 34 may be a single-use battery that requires replacement after some length of time, or a rechargeable battery that the user recharges by placing the system 10 in a charging cradle. In addition, power source 34 may be incorporated into the body 20 such that the power source 34 can not be seen but is easily replaced or recharged.

The display 36 may be an LCD, LED or any other type of display. The display 36 may be integrated into the holder 20 such that a user is able to view information which appears on the display 36.

A user may operate the example system 10 by manipulating the switch 32 to an “on” position. The user positions the sensor 22 near, or in, their mouth such that the sensor 22 collects molecules related to oral malodor (e.g., hydrogen sulfide, dimethyl sulfide, methyl mercaptan, and ethyl mercaptan) and/or harmful bacteria (e.g., gingivitis bacteria). The sensor 22 provides information to the display 36 regarding chemical and bacterial levels that are present in the user's mouth or on the user's breath.

This information may indicate the presence of oral malodor, plaque or tartar-causing substances, or bacteria that causes gingivitis and/or periodontal disease. In some embodiments, the information may also indicate the presence of cancer or viral infections (e.g., herpes simplex). It should be noted that the sensor 22 may detect relative concentrations of one or more substances.

As an example, display 36 may indicate a “malodor scale” that ranges from one to ten based on the sulfur level detected on the user's breath. A malodor level of one may indicate acceptable breath while a level of ten may indicate unacceptable breath.

As another example, display 36 may indicate a “bacterial scale” that ranges from one to ten. A bacterial level of one may indicate that there is a minimum level of harmful gingivitis bacteria which are present in the mouth while a level of ten may indicate that there is an unhealthy level of harmful gingivitis bacteria in the mouth.

In the example embodiment shown in FIG. 2, switch 32, power source 34 and display 36 are incorporated into electronic circuitry 50 that includes the sensor 22, a constant voltage generator circuit 52, a control circuit 54, an input network 56 and an output network 58. The sensor 22 may further include a first electrode 60, a second electrode 61 and a heating element 62. Heating element 62 is connected across power source 34 while sensor 22 is connected across power source 34 through control circuit 54.

The control circuit 54 is responsive to a variable differential voltage produced between the first electrode 60 and the second electrode 61 as the resistance R or conductance G varies across sensor 22. The control circuit 54 is coupled to an input network 64 that supplies the control circuit 54 with data signals during operation of the system 10. The control circuit 54 is also coupled to an output network 58 that receives signals from the control circuit 54 and then delivers output signals to the display 36.

During operation of the system 10, sensor 22 is thermally activated using the heating element 62. The system 10 is then exposed to malodorant gases. The resistance R or conductance G between the first electrode 60 and the second electrode 61 vary in proportion to the concentration of the malodorant substances. Therefore, a signal voltage, which is in proportion to the concentration of the detected malodorant gases, is produced between the first electrode 60 and the second electrode 61. The signal voltage is then supplied to control circuit 54 which delivers an output signal to the display 36 via output network 58.

In some embodiments, a constant voltage generator circuit 52 applies a voltage to the heating element 62 when the switch 32 is in the “on” position for the detection of malodorant gases. In addition, constant voltage generator circuit 52 may also apply a voltage to the heating element 62 when the switch is in the “off” position in order to regenerate the sensor 22. The “off” voltage may be much higher than the “on” voltage such that the heating element 62 bums away the molecules that were collected by the sensor 22.

In alternative embodiments of the system 10, the sensor 22 may be a polymer that collects specific molecules related to oral health and then changes color as target molecules are collected. In some forms, the intensity of the color is proportional to the number of target molecules that are collected.

As an example, sensor 22 may be designed to collect molecules related to oral malodor (e.g., sulfur). The sensor 22 may transition from white to red as the sensor 22 collects the relevant molecules. As the sensor 22 detects more relevant molecules, the intensity of the red color increases to provide a user with a relative measure as to the extent of the oral malodor.

It should be noted that the color change within the sensor 22 may indicate relative chemical and/or bacterial levels in a user's mouth or on the user's breath. In some embodiments, an individual may be able to compare the color of the sensor 22 to a printed color chart (e.g., printed on the holder 20) such that the user is able to determine the levels of substances which are present in the mouth.

As an example, sensor 22 may indicate a scale that ranges from light pink to deep red based on the sulfur level detected on the user's breath. A light pink color indicates acceptable breath and a deep red color indicates unacceptable breath.

As another example, sensor 22 may indicate a scale that ranges from light blue to deep blue. A light blue color may indicate that an acceptable level of harmful gingivitis bacteria is present in the mouth and a deep blue may indicate that an unhealthy level of harmful gingivitis bacteria is present in the mouth.

In some embodiments, sensor 22 may completely release the collected molecules when the system 10 is moved away from the mouth such that the sensor 22 returns to its original color. The amount of time that it takes for the sensor to regenerate will depend in part on the bacterial and/or chemical levels that were detected by sensor 22.

Once the sensor 22 has regenerated, a user may again be able to use the system 10 as described above in order to detect the presence of undesirable substances in the mouth. In other embodiments, a portion of the system 10 may be stored or treated in water (or some other liquid) that actively removes the malodorant substances which are collected by the sensor 22.

It should be noted that sensor 22 may be any type of sensor that is known now or discovered in the future. The type of sensor 22 that is used the system 10 will depend in part on the application where the system 10 is to be used.

In addition, oral treatment 24 may be any type of oral treatment that is known now or discovered in the future. One example oral treatment 24 may be a breath freshener that is used to treat oral malodor and/or plaque or tartar-causing substances. Another example oral treatment 24 may be used to treat bacteria that causes gingivitis and/or periodontal disease.

In some embodiments, the oral treatment 24 may treat cancer or viral infections (e.g., herpes simplex). It should be noted that the oral treatment 24 may provide one or more types of treatment. The type of oral treatment 24 that is used in the system 10 will depend in part on the application where the system 10 is to be used.

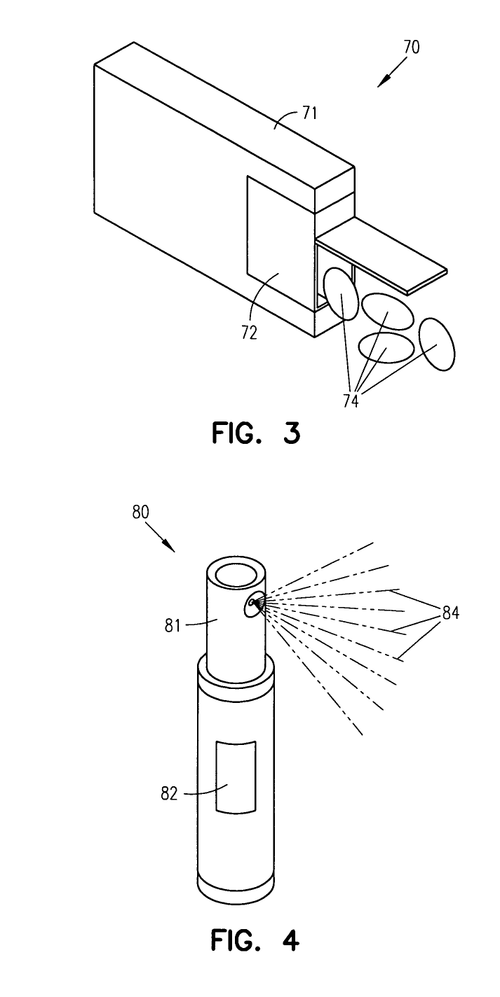

FIG. 3 shows another example system 70 of the present invention. The system 70 includes a holder (e.g., dispenser 71) and a sensor 72 that is attached to the dispenser 71. The system further includes an oral treatment 74 that is secured by the dispenser 71.

In the example embodiment illustrated in FIG. 4, the oral treatment 74 is a plurality of capsules (e.g., mints). It should be noted that the oral treatment 74 may take other forms besides capsules (e.g., pills, disks or spheres).

The sensor 72 may be any type of sensor that detects substances which influence oral health. Individuals may use the system 70 by exhaling a number of times on the sensor 72 and/or by engaging the sensor 72 with portions of their mouth or saliva in their mouth.

Another example system 80 of the present invention is shown in FIG. 4. The system 80 includes a holder (e.g., an aerosol sprayer 81) and a sensor 82 that is attached to the aerosol sprayer 81. The system 80 further includes an oral treatment 84 that is stored within the aerosol sprayer 81.

The sensor 82 may be any type of sensor that detects substances which influence oral health. Individuals may use the system 80 by spraying the oral treatment 84 into their mouth when the sensor 82 detects substances that influence oral health within their mouth.

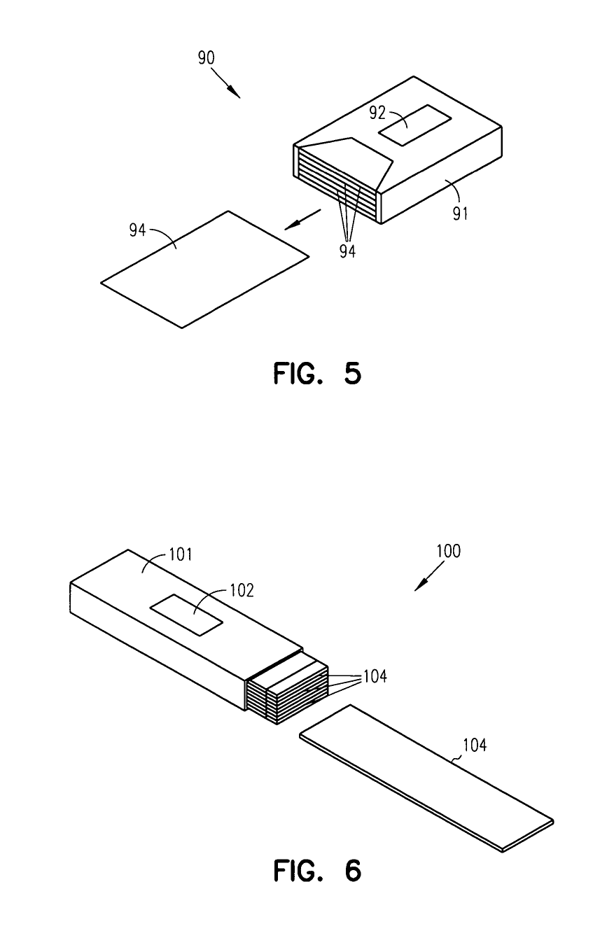

FIG. 5 illustrates another example system 90 of the present invention. The system 90 includes a holder 91 and a sensor 92 that is attached to the holder 91. The system 90 further includes an oral treatment (e.g., a plurality of breath strips 94). It should be noted that the breath strips 94 may be any type of breath strip that is known now or discovered in the future. In addition, the holder 91 may take any form as long as the holder 91 is able to distribute the breath strips 94 upon demand.

A user operates the system 90 by placing the sensor 92 within, or near, their mouth to collect molecules that are related to oral malodor (e.g., hydrogen sulfide, dimethyl sulfide, methyl mercaptan, and ethyl mercaptan) and/or harmful bacteria (e.g., gingivitis bacteria). In some embodiments, sensor 92 may detect relative concentrations of these substances. If the sensor 92 notifies the user that enough harmful substances have been detected, the user can remove a breath strip 94 from the holder 91 and then place the breath strip 94 into their mouth in order to treat their mouth.

FIG. 6 illustrates another example system 100 of the present invention. The system 100 includes a holder (e.g., package 101) and a sensor 102 that is attached to the package 101. The system further includes an oral treatment (e.g., sticks of gum 104) that are stored within the package 101.

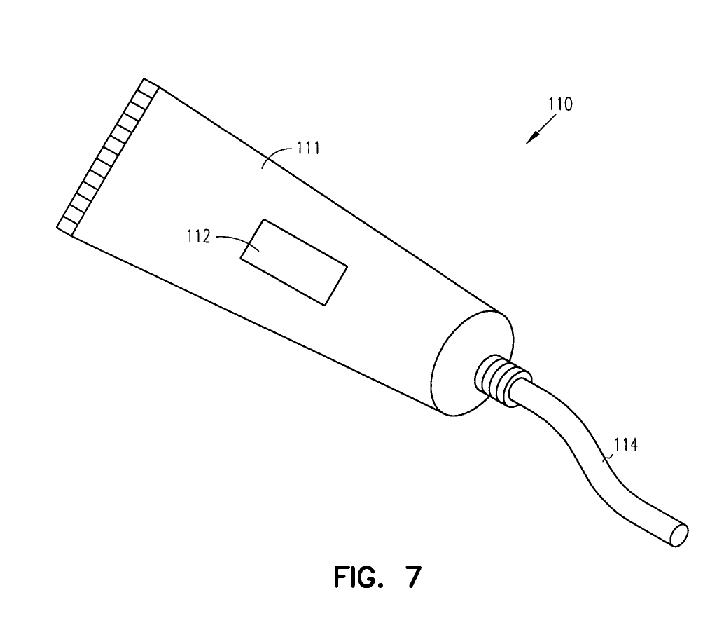

It should be noted that gum 104 may be any type of gum that is known now or discovered in the future. In the example embodiment shown in FIG. 7, the gum 104 is in the form of several sticks of gum that are stored within package 101, although the gum 104 may take any form. In addition, the gum 104 may be any flavor.

Sensor 102 may be similar to any of the sensors that are described or referenced above. The type of sensor 102 that is used the system 100 will depend in part on the application where the system 100 is to be used.

A user operates the system 100 by positioning the sensor 102 within, or near, a mouth to collect molecules that are related to oral malodor (e.g., hydrogen sulfide, acetone, ammonia, dimethyl sulfide, methyl mercaptan, and ethyl mercaptan) and/or harmful bacteria (e.g., gingivitis bacteria). If the sensor 102 indicates to the user that sufficient number of molecules have been detected, the user simply places a stick of gum 104 into their mouth in order to treat their mouth.

FIG. 7 illustrates another example system 110 of the present invention. The system 110 includes a holder (e.g., tube 111) and a sensor 112 that is attached to the tube 111. The system further includes an oral treatment (e.g., toothpaste 114) that is secured by the tube 111. It should be noted that tube 111 may be any type of holder that is able to conveniently dispense toothpaste 114.

The sensor 112 is adapted to detect substances that influence oral health when the system 110 is placed near a mouth. The sensor 112 may be integrated into the tube 111 such that the sensor 112 is exposed to a user's saliva and/or breath when the tube 111 is placed near, or within, the user's mouth.

In any of the embodiments described or references herein, the sensors may provide information as to a dosage of the oral treatment that should be applied to treat the mouth. The information may be provided in any form.

As an example, the sensor may provide alphanumeric information (e.g., 250 ml or 2 capsules). As another example, the sensor may change color such that the sensor can be matched to a color chart that provides dosage information.

It should be noted that with regard to any of the sensors described herein, the sensors may completely release the molecules that were collected during use such that the sensors return to an original state. The amount of time that it takes for the sensors to regenerate will depend in part on the bacterial and/or chemical levels that were detected by sensor.

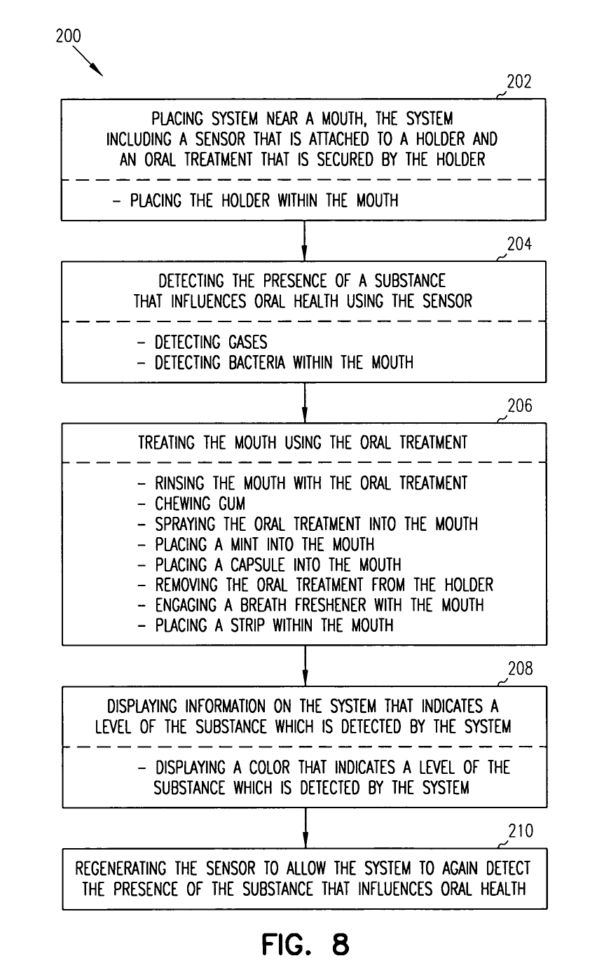

FIG. 8 illustrates an example method 200 of the present invention. The method 200 includes placing a system near a mouth 202 (e.g., system 10 shown in FIG. 1). The system includes a holder and a sensor that is attached to the holder. The system further includes an oral treatment that is secured by the holder. The method 200 further includes detecting the presence of a substance that influences oral health using the sensor 204 and treating the mouth using the oral treatment 206.

In some embodiments, placing the system near the mouth 202 may include placing the holder within the mouth. It should be noted that detecting the presence of the substance that influences oral health 204 may include detecting gases that are within (or exhaled from) the mouth and/or detecting bacteria that are within the mouth.

The method 200 may further include displaying information on the system that indicates a level of the substance which is detected by the system 208. Displaying information on the system 208 may include displaying a color that indicates the level of the substance which is detected by the system. In some embodiments, the displayed color may be compared to a chart to determine the level of the substance that is within the mouth.

In some embodiments, displaying information on the system 208 may include displaying a dosage of the oral treatment that should be applied to treat the mouth. As examples, the dosage-related information may be displayed alphanumerically or a displayed color may be compared to a chart to determine an appropriate dosage amount.

The method 200 may further include regenerating the sensor to allow the system to again detect the presence of the substance that influences oral health 210. As discussed above, the amount of time that it takes for the sensor to regenerate may depend in part on the level of the substance that was detected by sensor.

In some embodiments, treating the mouth using the oral treatment 206 may include (i) rinsing the mouth with the oral treatment; (ii) chewing gum; (iii) spraying the oral treatment into the mouth; (iv) placing a mint into the mouth; (v) placing a capsule, pill, disk and/or sphere into the mouth; (vi) removing the oral treatment from the holder; (vii) engaging a breath freshener with the mouth; and/or (viii) placing a strip within the mouth.

It should be noted that any of the systems and methods described herein may be able to indicate the presence of harmful bacterial and/or oral malodor in the mouth and have the ability to treat the mouth. In addition, the systems and methods may be configured to detect pH levels or other oral health parameters and to provide a variety of types of oral treatment.

Any of the systems that are shown and/or described herein may be used in any of the methods that are described or referenced herein. While the invention has been described in detail with respect to specific embodiments, it will be appreciated that there are variations of, and equivalents to these embodiments. Accordingly, the scope of the present invention should be determined by the appended claims and any equivalents thereto.

1-36. (canceled)

37. A system comprising:
a holder;
a sensor attached to the holder, the sensor being adapted to detect a gas that influences oral health when the system is placed near a mouth; and
an oral treatment secured by the holder, the oral treatment being adapted to treat the mouth when the oral treatment engages the mouth.

38. The system of claim 37 wherein the oral treatment is a mouthwash.

39. The system of claim 37 wherein the holder is a package.

40. The system of claim 37 wherein the oral treatment is a capsule.

41. The system of claim 37 wherein the oral treatment is a gum.

42. The system of claim 37 wherein the sensor detects the substance that influences oral health when the system is placed within the mouth.

43. The system of claim 37 wherein the sensor is detachably connected to the holder.

44. The system of claim 37 wherein the holder includes an aerosol sprayer and the oral treatment is a liquid that is sprayed from the aerosol sprayer.

45. The system of claim 37 wherein the oral treatment is a breath strip.

46. A system comprising:
a holder;
a sensor attached to the holder, the sensor being adapted to detect a substance that influences oral health when the system is placed near a mouth; and
an oral treatment secured by the holder, the oral treatment being adapted to treat the mouth when the oral treatment engages the mouth, wherein the sensor provides information as to a dosage of the oral treatment that should be applied to treat the mouth.

47. The system of claim 46 wherein the oral treatment is a mouthwash.

48. The system of claim 46 wherein the holder is a package.

49. The system of claim 46 wherein the oral treatment is a capsule.

50. The system of claim 46 wherein the oral treatment is a gum.

51. The system of claim 46 wherein the sensor detects the substance that influences oral health when the system is placed within the mouth.

52. The system of claim 46 wherein the sensor is detachably connected to the holder.

53. The system of claim 46 wherein the holder includes an aerosol sprayer and the oral treatment is a liquid that is sprayed from the aerosol sprayer.

54. The system of claim 46 wherein the oral treatment is a breath strip.

55. A method comprising:
placing a system near a mouth, the system including a sensor that is attached to a holder and an oral treatment that is secured by the holder;
detecting the presence of a gas that influences oral health using the sensor; and
treating the mouth using the oral treatment.

56. The method of claim 55 wherein treating the mouth using the oral treatment includes rinsing the mouth with the oral treatment.

57. The method of claim 55 wherein treating the mouth using the oral treatment includes chewing gum.

58. The method of claim 55 wherein treating the mouth using the oral treatment includes spraying the oral treatment into the mouth.

59. The method of claim 55 wherein treating the mouth using the oral treatment includes placing a capsule into the mouth.

60. The method of claim 55 wherein treating the mouth using the oral treatment includes placing a breath strip within the mouth.

61. A method comprising:
placing a system near a mouth, the system including a sensor that is attached to a holder and an oral treatment that is secured by the holder;
detecting the presence of a substance that influences oral health using the sensor;
displaying information on the system that relates to a dosage of the oral treatment which should be applied to treat the mouth based on an amount of the substance that is detected by the sensor; and
treating the mouth using the oral treatment.

62. The method of claim 61 wherein treating the mouth using the oral treatment includes rinsing the mouth with the oral treatment.

63. The method of claim 61 wherein treating the mouth using the oral treatment includes chewing gum.

64. The method of claim 61 wherein treating the mouth using the oral treatment includes spraying the oral treatment into the mouth.

65. The method of claim 61 wherein treating the mouth using the oral treatment includes placing a breath strip within the mouth.
